(19) United States
(12) Patent Application Publication
(10) Pub. No.: US 2026/0007812 A1
(43) Pub. Date: Jan. 8, 2026

(54) AUTOMATED PERITONEAL DIALYSIS SYSTEM HAVING MACHINE DOCKING STATION

(71) Applicants: VANTIVE US HEALTHCARE LLC, Deerfield, IL (US); VANTIVE HEALTH GMBH, Glattpark (Opfikon) (CH)

(72) Inventors: Andreas WINQUIST, Lund (SE); Sven GUSTAFSON, Lund (SE); Peter John DRENNAN, Lund (SE); Matthew FOURIE, Lund (SE)

(21) Appl. No.: 19/133,934

(22) PCT Filed: Nov. 28, 2023
(86) PCT No.: PCT/US2023/081403

§ 371 Date: May 29, 2025

Related U.S. Application Data
(60) Provisional application No. 63/428,559, filed on Nov. 29, 2022.

(51) Int. Cl. A61M 1/28; A61M 1/36

(52) U.S. Cl. CPC A61M 1/28; A61M 1/369; A61M 2205/3584; A61M 2205/52; A61M 2205/70; A61M 2205/8262

(57) ABSTRACT

A peritoneal dialysis (“PD”) system includes a PD machine and a docking station connected to the PD machine via a power cord. The docking station includes a plurality of connectors configured to receive a cable of one or more wired peripheral entity, a transceiver for wireless communication with one or more wireless peripheral entity, and a power supply rated to power the PD machine during a PD treatment that includes batch or inline PD fluid heating.

### TECHNICAL FIELD

The present disclosure relates generally to medical fluid treatments, and in particular to dialysis fluid treatments.

### BACKGROUND

Due to various causes, a person's renal system can fail. Renal failure produces several physiological derangements. It is no longer possible to balance water and minerals or to excrete a daily metabolic load. Toxic end products of metabolism, such as, urea, creatinine, uric acid, and others, may accumulate in a patient's blood and tissue.

Reduced kidney function and, above all, kidney failure is treated with dialysis. Dialysis removes waste, toxins, and excess water from a body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across a semi-permeable dialyzer between blood and an electrolyte solution, called dialysate or dialysis fluid, to cause diffusion.

Hemofiltration (“HF”) is an alternative renal replacement therapy that relies on a convective transport of toxins from a patient's blood. HF is accomplished by adding substitution or replacement fluid to an extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from a patient's home, causing door-to-door treatment time to consume a large portion of the day. Treatments in centers close to the patient's home may also consume a large portion of the patient's day. HHD can take place overnight or during the day while the patient relaxes, works, or is otherwise productive.

Another type of kidney failure therapy is peritoneal dialysis (“PD”), which infuses a dialysis solution, also called dialysis fluid, into a patient's peritoneal chamber via a catheter. The dialysis fluid contacts a peritoneal membrane in the patient's peritoneal chamber. Waste, toxins, and excess water pass from the patient's bloodstream, through the capillaries in the peritoneal membrane, and into the dialysis fluid due to diffusion and osmosis, i.e., an osmotic gradient occurs across the membrane. An osmotic agent in the PD fluid provides the osmotic gradient. Used or spent dialysis fluid is drained from the patient, thereby removing waste, toxins, and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, where the transfer of waste, toxins, and excess water takes place. After a dwell period, the patient repeats the manual dialysis procedure, for example, four times per day. Manual peritoneal dialysis requires a significant amount of time and effort from the patient, leaving ample room for improvement.

APD is similar to CAPD in that the dialysis treatment includes drain, fill, and dwell cycles. PD machines, however, perform the cycles automatically, typically while the patient sleeps. PD machines free patients from having to manually perform the treatment cycles and from having to transport supplies during the day. PD machines connect fluidly to an implanted catheter, a source or bag of fresh dialysis fluid, and a fluid drain. PD machines pump fresh dialysis fluid from a dialysis fluid source, through the catheter, and into the patient's peritoneal chamber. PD machines also allow for the dialysis fluid to dwell within the chamber and for the transfer of waste, toxins, and excess water to take place. The source may include multiple liters of dialysis fluid including several solution bags.

PD machines pump used or spent dialysate from the patient's peritoneal cavity, though the catheter, and to the drain. As with the manual process, several drain, fill, and dwell cycles occur during dialysis. A “last fill” may occur at the end of the PD treatment. The last fill fluid may remain in the peritoneal chamber of the patient until the start of the next treatment, or may be manually emptied at some point during the day.

Any of the above renal failure therapy machines, but especially PD machines, are provided in different versions and/or varieties. Different markets have different requirements resulting in different versions of the same PD machine. Also, new PD machines are developed from time to time in an attempt to improve upon older PD machines. Nevertheless, the older PD machines remain on the market because PD machines typically last a long time and may be preferred by certain patients or clinicians who have grown to be very familiar with the older PD machines. Adding across-the-board updates and functionality to the different PD machines and different versions of the same PD machine has proven to be problematic. The different machines and different machine versions may have different processors and memories and have different types and sophistication levels of connectivity, e.g., may or may not have wireless communication capability.

For the above reasons, an improved PD system and associated methodology is needed.

### SUMMARY

The present disclosure sets forth an automated peritoneal dialysis (“PD”) system and associated methodology, where PD machines operate with a docking station. The docking station provides the appropriate power and data communication to the PD machine or cycler. In an embodiment, a power cable is the only line from the docking station to the PD machine. The cable is, in one embodiment, a multi-conductor or wire cable, which includes a power conductor (e.g., multiple stranded wires) and a data conductor. The PD machine or cycler includes a connector or port configured to receive the multi-conductor or wire cable and to communicate electrically separately with the power conductor and the data conductor. In this manner, power carried by the power conductor may pass through needed power conversion circuitry (transformers, rectifies, etc.) within the PD machine, while data carried by the data conductor is delivered to/from a control unit of the PD machine. It should be appreciated however that the docking station of the present disclosure is not limited to PD machines having connectors or ports configured to receive a multi-conductor or wire cable. Embodiments of the docking station operating with older such PD machines are discussed herein.

The docking station includes a plurality of connectors/ports, such as one or more of universal serial bus (“USB”, including USB-A, USB-B, USB-C, USB 3.0) connectors/ports, high-definition multimedia (“HDMI”) connectors/ports, RCA connectors/ports, 3.5 mm audio connectors/ports, Ethernet connectors/ports, SD card reader connectors/ports, micro SD reader connectors/ports, displayport connectors/ports, Deutsches Institut fur Normung (“DIN”) connectors, including all types of DIN connectors, D connectors, and new connectors for new or existing communication protocols developed in the future. The connectors/ports are connected electrically to a control unit of the docking station. The control unit includes one or more processor, one or more memory, and a transceiver for two-way wireless data communication between the control unit and any wireless peripheral entity/device operating with the docking station.

The docking station is configured to be connected to many different types of peripheral entities, such as blood pressure monitors, weight scales, glucose monitors, oxygen monitors, and other medical monitors, the Internet, local and wide area networks, smartphones, and cellphones. Any of the peripheral entities may be wirelessly connected to the docking station. Certain of the peripheral entities may be placed in wired communication with the docking station, such as, blood pressure monitors, weight scales, glucose monitors, oxygen monitors, and other medical monitors.

Certain peripheral entities, such as blood pressure monitors, weight scales, glucose monitors, oxygen monitors, and other medical monitors, operate with the docking station without the docking station interacting with the PD machine, i.e., independently of the communication between the docking station and the PD machine or cycler. The docking station here stands alone with the peripheral entity, which in one embodiment operates without the PD machine even knowing about the interaction. When a medical monitor is connected in a wired manner to the docking station, the medical monitor in a plug and play (“PnP”) configuration in one embodiment has its own software and provides drivers so that the control unit of the docking station is able to automatically and dynamically recognize the type of medical monitor (e.g., blood pressure, weight scale, glucose, oxygen) and assign that medical monitor to a corresponding connector/port of the docking station. The PnP medical monitor also loads the needed one or more drivers into the control unit of the docking station, thereby enabling the docking station to interact appropriately with the particular type of medical monitor data being supplied to the docking station.

When the medical monitor is connected in a wireless manner to the docking station, the medical monitor first undergoes a pairing routine such that the output from the medical monitor is only transmitted to the correct docking station, e.g., in a clinical setting where there may be multiple PD machines and docking stations. The pairing may occur by placing a “PAIR” button on the docking station. When the button is pressed, the transceiver of the docking station sends a signal, which the wireless medical monitor recognizes and synchs to. Thereafter, any and all outputs from the wireless medical monitor are sent to the paired docking station.

Second, the appropriate drivers and wireless medical monitor software needed to operate with the wireless medical monitor are sent wirelessly to the docking station. To do so, a software application associated with the wireless medical monitor is downloaded onto a user's smartphone, tablet, or other smart device. The docking station and the smart device both connect to an available wireless network, e.g., WiFi, in one embodiment. The wireless network, e.g., WiFi, enables the appropriate drivers and wireless medical monitor software to be downloaded to the control unit of the docking station from the application software associated with the wireless medical monitor. In an alternative embodiment, the application software is downloaded wirelessly to the docking station directly from the medical monitor. In any case, the appropriate drivers and wireless medical monitor software inform the control unit of the docking station as to the type of medical monitor, e.g., blood pressure, weight scale, glucose, oxygen, etc. The drivers and wireless medical monitor software also enable the docking station to interact appropriately with the particular type of medical monitor data being supplied to the docking station. The above-described downloading of the software application may be performed alternatively in a wired manner, e.g., by connecting a memory stick or flash drive to one of the ports on the docking station.

The connection of the docking station to the available wired or wireless network, e.g., WiFi, also enables the docking station to access the Internet. Through the Internet, the control unit of the docking station of the present disclosure is able to access a remote medical data acquisition and analysis connectivity platform, such as the Sharesource™ platform provided by the assignee of the present disclosure. The remote medical data acquisition and analysis connectivity platform enables the control unit of the docking station to connect with relevant clinician and/or hospital systems. The remote medical data acquisition and analysis connectivity platform also provides data logging sheets or pages, which are populated with medical treatment/monitoring data from the docking station, and which are accessible by the relevant clinician and/or hospital systems for clinician and/or doctor review.

The clinician, based on the medical monitoring data, may determine that one or more of the patient's PD treatment prescriptions needs to be updated or perhaps replaced. The remote medical data acquisition and analysis connectivity platform allows the updated or replaced PD treatment prescription to be delivered to the control unit of the docking station. Delivering the updated or replaced PD treatment prescription to a control unit of the PD machine from the docking station depends upon the age and communication capabilities of the PD machine.

Many older PD machines, which are still in use and which are still manufactured and sold as new machines, do not have wired or wireless connection capability. Most of those PD machines, however, have a data port for receiving a memory stick or flash drive (or other removable memory device) to download treatment data to, and receive new treatment prescription data from, the memory stick or a flash drive. In the past, the PD patient had to transport the memory stick or the flash drive with treatment data to a hospital or clinic, where the doctor or clinician used the memory stick or the flash drive to upload treatment data to their system, make an updated or new PD treatment prescription, save the updated or new PD treatment prescription to the memory stick or the flash drive, where the patient then transported the memory stick or the flash drive back to the PD machine to install the updated or new treatment prescription.

The docking station of the present disclosure instead resides adjacent to the PD machine. The patient only has to insert the memory stick, the flash drive, or other removable memory device into the docking station, download the updated or new treatment prescription onto the memory stick or the flash drive, and plug the memory stick or the flash drive into the adjacent PD machine to install the updated or new treatment prescription. Likewise, treatment data may be downloaded from the control unit of the PD machine to the memory stick or the flash drive, which is then plugged into the adjacent docking station, where the treatment data is uploaded to the control unit of the docking station, which may then be transported via the remote medical data acquisition and analysis connectivity platform to the appropriate doctor's or clinician's system.

Newer PD machines are provided with wireless capability, which allows the control units of the newer PD machines to either (i) connect via the Internet directly to the remote medical data acquisition and analysis connectivity platform to share updated or new treatment prescriptions (to the PD machine) and treatment data (to the doctor or clinician) with the doctor's or clinician's system or (ii) connect wirelessly with the docking station, which in turn connects via the Internet to the remote medical data acquisition and analysis connectivity platform to share updated or new treatment prescriptions (to the PD machine) and treatment data (to the doctor or clinician) with the doctor's or clinician's system.

It is also contemplated for the docking station to be configured to send and/or relay text messages (SMS or MMS messages), e.g., from a doctor's or clinician's cellphone to the patient. In another example, the docking station is configured to, under certain treatment or PD machine conditions, relay a wired or wireless alarm sent from the PD machine to the docking station out to the doctor or clinician cellphone via an SMS or MMS message and/or to a service technician for the PD machine or cycler. Moreover, altering a doctor, clinician, and/or technician to a certain treatment or machine, e.g., alarm, condition is expressly not limited to SMS or MMS messaging and may be done via other wired or wireless communication protocols.

The docking station of the present disclosure enables an existing PD machine to be used, with its existing hardware and connectivity, in a system having new advancements in any one or more of (i) medical monitoring, (ii) PD treatment procedures, (iii) data communication, and (iv) data connectivity. Different docking stations may be provided that are customized for the particular age, capability, and regional requirements of or for the PD machine. Additionally, new and updated docking stations may be provided as technology advances, while allowing existing and patient familiar cyclers to continue to be used.

In light of the disclosure set forth herein, and without limiting the disclosure in any way, in a first aspect, which may be combined with any other aspect or portion thereof, a peritoneal dialysis (“PD”) system includes a PD machine and a docking station connected to the PD machine via a power cord, where the power cord is configured to bring power and optionally data to the PD machine. The docking station includes a plurality of connectors configured to receive a cable of one or more wired peripheral entity, a transceiver for wireless communication with one or more wireless peripheral entity, and a power supply rated to supply enough power to power the PD machine during a PD treatment, the PD treatment including batch or inline PD fluid heating.

In a second aspect of the present disclosure, which may be combined with any other aspect or portion thereof, the power cord is the only physical connection between the PD machine and the docking station.

In a third aspect of the present disclosure, which may be combined with any other aspect or portion thereof, the PD system is configured such that the PD machine is connectable to a plurality of different versions or varieties of the docking station.

In a fourth aspect of the present disclosure, which may be combined with any other aspect or portion thereof, the PD system further includes the one or more wired and wireless peripheral devices, including at least one medical monitor, the Internet, a network, a smart device, or a cellphone.

In a fifth aspect of the present disclosure, which may be combined with any other aspect or portion thereof, the PD machine and the docking station each includes a computer memory connector. A control unit of the PD machine is in communication with the PD machine computer memory connector and a control unit of the docking station is in communication with the docking station computer memory connector. Additionally, a memory stick is used to transport data between the control unit of the PD machine and the control unit of the docking station via the respective computer memory connectors.

In a sixth aspect of the present disclosure, which may be combined with any other aspect or portion thereof, the data includes PD treatment data from the PD machine and one or more PD treatment prescription from the docking station.

In a seventh aspect of the present disclosure, which may be combined with any other aspect or portion thereof, the PD system further includes a clinician and/or hospital system. Additionally, the docking station is configured to (i) transmit PD treatment data to the clinician and/or hospital system and (ii) receive the one or more PD treatment prescription from the clinician and/or hospital system.

In an eighth aspect of the present disclosure, which may be combined with any other aspect or portion thereof, the docking station is configured to (i) transmit to and (ii) receive from the clinician and/or hospital system via at least one of the Internet or a data acquisition and analysis connectivity platform.

In a ninth aspect of the present disclosure, which may be combined with any other aspect or portion thereof, the PD machine is configured to communicate wirelessly with the docking station to transport data between the PD machine and the docking station.

In a tenth aspect of the present disclosure, which may be combined with any other aspect or portion thereof, the data includes PD treatment data from the PD machine and one or more PD treatment prescription from the docking station.

In an eleventh aspect of the present disclosure, which may be combined with any other aspect or portion thereof, the PD system further includes a clinician and/or hospital system. Additionally, the docking station is configured to (i) transmit PD treatment data to the clinician and/or hospital system and (ii) receive the one or more PD treatment prescription from the clinician and/or hospital system.

In a twelfth aspect of the present disclosure, which may be combined with any other aspect or portion thereof, the PD system further includes a clinician and/or hospital system, Additionally, the PD machine is configured to communicate wirelessly with the clinician and/or hospital system to transport data between the PD machine and the clinician and/or hospital system.

In a thirteenth aspect of the present disclosure, which may be combined with any other aspect or portion thereof, the data includes PD treatment data from the PD machine and one or more PD treatment prescription from the clinician and/or hospital system.

In a fourteenth aspect of the present disclosure, which may be combined with any other aspect or portion thereof, the PD machine is configured to communicate wirelessly with the clinician and/or hospital system via at least one of the Internet or a data acquisition and analysis connectivity platform.

In a fifteenth aspect of the present disclosure, which may be combined with any other aspect or portion thereof, the PD system further includes a smart service connector configured to be connected to the PD machine while waiting at a service center for servicing, the smart service connector configured to identify at least one action or fix needed for the PD machine prior to commencing the servicing.

In a sixteenth aspect of the present disclosure, which may be combined with any other aspect or portion thereof, the smart service connector is configured to be plugged into a computer memory connector of the PD machine.

In a seventeenth aspect of the present disclosure, which may be combined with any other aspect or portion thereof, the smart service connector is configured to cause at least one component of the PD machine to be actuated to test the at least one component.

In an eighteenth aspect of the present disclosure, which may be combined with any other aspect or portion thereof, the smart service connector includes a rechargeable battery configured to be recharged by the PD machine.

In a nineteenth aspect of the present disclosure, any of the features, functionality and alternatives described in connection with any one or more of FIGS. 1 to 3 may be combined with any of the features, functionality and alternatives described in connection with any other of FIGS. 1 to 3 and/or any of the aspects listed herein.

It is accordingly an advantage of the present disclosure to provide a peritoneal dialysis system having a universal docking station that is connectable and useable with different PD machines and different versions of PD machines.

It is another advantage of the present disclosure to provide a PD system that adapts to new technology and functionality without having to change or upgrade the PD machine.

It is a further advantage of the present disclosure to provide a PD system that further prolongs the life cycle of a PD machine.

It is yet another advantage of the present disclosure to provide a PD system that allows for different functionalities, e.g., for different markets and regional areas, without having to provide different versions of the same PD machine.

It is yet a further advantage of the present disclosure to provide a PD system that more readily adapts to changes in data communication and protocols.

Moreover, it is an advantage of the present disclosure to provide a system having a PD machine and docking station with only one cord or cable connected between the PD machine and the docking station.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic view of one embodiment of a system of the present disclosure, which includes a PD machine, a docking station, a remote medical data acquisition and analysis connectivity platform, and a relevant clinician and/or hospital system.

FIG. 2 is a perspective view of one embodiment of a docking station of the present disclosure.

FIG. 3 is a schematic view of one embodiment for a smart service connector operable with a PD machine while at a service center or depot.

### DETAILED DESCRIPTION

### System Overview

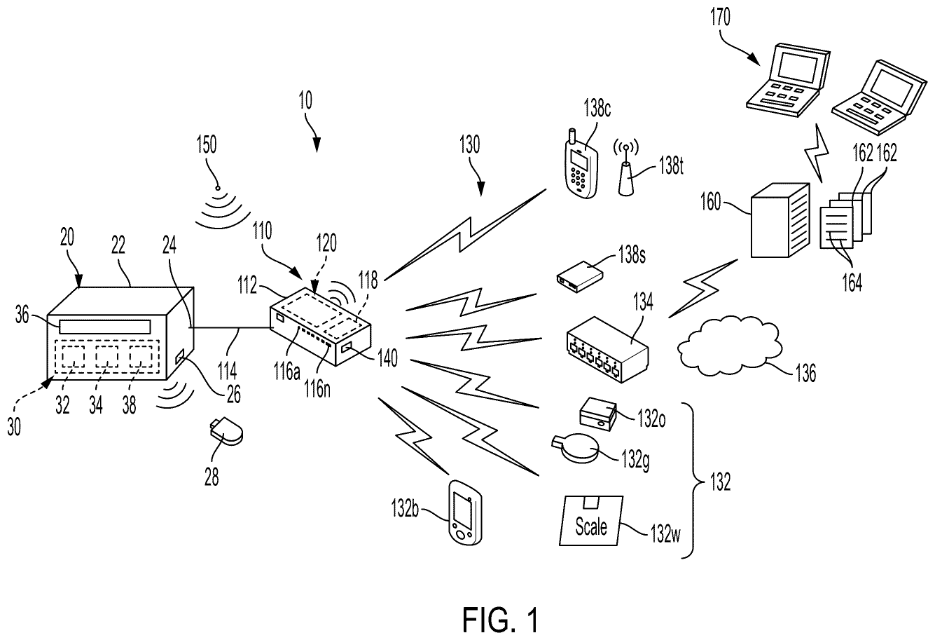

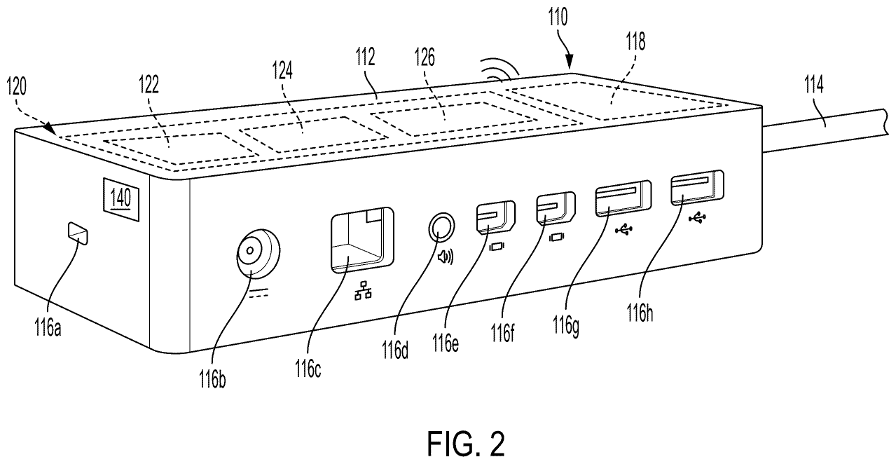

Referring now to the drawings and in particular to FIGS. 1 and 2, the present disclosure sets forth an automated peritoneal dialysis (“PD”) system 10 and associated methodology, where a PD machine 20 operates with a docking station 110. PD machine 20 includes a housing 22, while docking station 110 includes a housing 112. In various embodiments, all rigid and flexible components of housings 22 and 112 may be made of one or more plastic, e.g., polyvinylchloride (“PVC”) or a non-PVC material, such as polyethylene (“PE”), polyurethane (“PU”), polycarbonate (“PC”) and/or polyetheretherketone (“PEEK”). Housings 22 and 112 may alternatively or additionally be made of any one or more metal, e.g., stainless steel, steel, and/or aluminum. In the illustrated embodiment, docking station 110 supplies the appropriate power to PD machine 20. In an embodiment, a power cable or cord 114 is the only line from docking station 110 to PD machine 20.

Cable or cord 114 is in one embodiment a multi-conductor or wire cable or cord, which includes a power conductor (e.g., multiple stranded wires) and a data conductor. PD machine or cycler 20 includes a connector or port 24 configured to receive the multi-conductor or wire cable or cord 114 and to communicate electrically separately with the power conductor and the data conductor. In this manner, power carried by the power conductor may pass through needed power conversion circuitry (transformers, rectifies, etc.) within PD machine 20, while data carried by the data conductor is delivered to/from a control unit 30 of the PD machine 20. It should be appreciated however that docking station 110 of the present disclosure is not limited to PD machines having connectors or ports configured to receive a multi-conductor or wire cable. Embodiments of docking station 110 operating with older such PD machines 20 are discussed herein.

Docking station 110 includes a plurality of ports or connectors 116a to 116n for receiving wires or cables from peripheral devices 130, such as one or more of universal serial bus (“USB”, including USB-A, USB-B, USB-C, USB 3.0) ports/connectors, high-definition multimedia (“HDMI”) ports/connectors, RCA ports/connectors, 3.5 mm audio ports/connectors, Ethernet ports/connectors, SD card reader ports/connectors, micro SD reader ports/connectors, displayport ports/connectors, Deutsches Institut fur Normung (“DIN”) connectors, including all types of DIN connectors, D connectors, and new connectors for new or existing communication protocols developed in the future. Ports or connectors 116a to 116n may, for example, have power ratings up to 70 Amps. Ports or connectors 116a to 116n are provided in one embodiment with the ability to transfer signals and data together with power along the connected line in the same connector. Ports or connectors 116a to 116n are connected electrically to a control unit 120 of docking station 110. Control unit 120 includes one or more processor 122, one or more memory 124, and a transceiver 126 for two-way wireless data communication between control unit 120 and any wireless peripheral device operating with docking station 110.

Docking station 110 is configured to be connected (wired to ports or connectors 116a to 116n, or wirelessly) to many different types of peripheral entities 130, such as blood pressure monitors 132b, weight scales 132w, glucose monitors 132g, oxygen monitors 1320, and other medical monitors 132, the Internet 134, local and wide area networks 136, smartphones and other smart devices (tablets, etc.) 138s, and cellphones 138c (e.g., via a cellphone tower 138t). Any of peripheral entities 130 may be wirelessly connected to docking station 110. Certain peripheral entities 130 may be placed in wired or wireless communication with docking station 110, such as, blood pressure monitors 132b, weight scales 132w, glucose monitors 132g, oxygen monitors 1320, other medical monitors 132, the Internet 134, and local and wide area networks 136.

Certain peripheral entities 130, such as blood pressure monitors 132b, weight scales 132w, glucose monitors 132g, oxygen monitors 1320, and other medical monitors 132, operate with docking station 110 without the docking station interacting with PD machine 20. Docking station 110 here stands alone with peripheral entity 130, which in one embodiment operates without PD machine 20 even knowing about the interaction. When a medical monitor 132 is connected in a wired manner to docking station 110, the medical monitor in a plug and play (“PnP”) configuration in one embodiment has its own software and provides drivers so that the control unit 120 of docking station 110 is able to automatically and dynamically recognize the type of medical monitor 132 (e.g., blood pressure 132b, weight scale 132w, glucose 132g, oxygen 1320) and assign that medical monitor to corresponding port 116a to 116n of docking station 110. The PnP medical monitor 132 also loads the needed one or more drivers into control unit 120 of docking station 110, so that the docking station is able to interact appropriately with the particular type of medical monitor data being supplied to the docking station.

When medical monitor 132 is connected in a wireless manner to docking station 110, the medical monitor first undergoes a pairing routine such that the output from the medical monitor is only sent to the correct docking station 110, e.g., in a clinical setting where there may be multiple PD machines 20 and docking stations 110. The pairing may take by including a “PAIR” button 140 on docking station 110. When “PAIR” button 140 is pressed, transceiver 126 of docking station 110 transmits a signal, which wireless medical monitor 132 recognizes and synchs to. Thereafter, any and all outputs from wireless medical monitor 132 are sent to the paired docking station 110.

Second, the appropriate drivers and wireless medical monitor software needed to operate with wireless medical monitor 132 are sent wirelessly to docking station 110. To do so, a software application associated with wireless medical monitor 132 is downloaded onto a user's smartphone, tablet, or other smart device 138s. Docking station 110 and smart device 138s both connect to an available wireless network 150, e.g., WiFi, in one embodiment. Wireless network 150, e.g., WiFi, enables the appropriate drivers and wireless medical monitor software to be downloaded to control unit 120 of docking station 110 from the application software associated with wireless medical monitor 132. In an alternative embodiment, the application software is downloaded wirelessly to docking station 110 directly from medical monitor 132. In any case, the appropriate drivers and wireless medical monitor software inform control unit 120 of docking station 110 as to the type of medical monitor 132, e.g., blood pressure 132b, weight scale 132w, glucose 132g, oxygen 1320, etc. The drivers and wireless medical monitor software also enable docking station 110 to interact appropriately with the particular type of medical monitor data being supplied to the docking station. The above-described downloading of the software application may be performed alternatively in a wired manner, e.g., by connecting a memory stick, flash drive or other removable memory device 28 to one of the ports or connectors 116a to 116n on docking station 110.

The connection of docking station 110 to the available wired or wireless network 150, e.g., WiFi, also enables docking station 110 to access the Internet 134. Through the Internet 134, control unit 120 of docking station 110 of system 10 is able to access a remote medical data acquisition and analysis connectivity platform 160, such as the Sharesource™ platform provided by the assignee of the present disclosure. The remote medical data acquisition and analysis connectivity platform 160 enables control unit 120 of docking station 110 to connect with the relevant clinician and/or hospital systems 170. The remote medical data acquisition and analysis connectivity platform 160 also provides data logging sheets or pages 162, which are populated with medical monitoring data from docking station 110, and which are accessible by the relevant clinician and/or hospital systems 170 for clinician or doctor review.

The clinician, based on the medical monitoring data, may determine that one or more of the patient's PD treatment prescriptions 164 needs to be updated or perhaps replaced. The remote medical data acquisition and analysis connectivity platform 160 allows one or more updated or replaced PD treatment prescription 164 to be delivered to control unit 120 of docking station 110. Delivering the one or more updated or replaced PD treatment prescription 164 to a control unit 30 of PD machine 20 from docking station 110 depends upon the age and communication capabilities of the PD machine. Control unit 30 of PD machine 20 varies depending on the age of the design of the PD machine and may include one or more processor 32, one or more memory 34, a user interface 36, and possibly, if new enough, a transceiver 38 for wireless two way communication with control unit 120 of docking station 110 and to remote entities accessed via the Internet 134.

Many older PD machines 20, e.g., ones not having a connector or port 24 capable of receiving a multi-conductor or wire version of cable or cord 114, which are still in use and which are still manufactured and sold as new machines, do not have wired or wireless connection capability. Most of the older PD machines 20, however, have a computer memory or data connector/port 26 for receiving a memory stick, flash drive, or other removable memory device 28 to download treatment data to, and receive new treatment prescription data from, the memory stick or flash drive. In the past, the PD patient had to transport the memory stick or flash drive 28 with treatment data to a hospital or clinic, where the doctor or clinician plugged the memory stick or flash drive 28 into their computer to upload treatment data from the stick to their system 170, form an updated or new PD treatment prescription 164 if needed, save the updated or new PD treatment prescription 164 to the memory stick or flash drive 28, where the patient then transported the memory stick or flash drive 28 back to PD machine 20 to install the updated or new treatment prescription 164.

Docking station 110 of the present disclosure instead resides adjacent to PD machine 20. The patient only has to insert the memory stick, flash drive, or other removable memory device 28 into docking station 110 storing the updated or new prescription 164, download the updated or new treatment prescription onto the memory stick or flash drive 28, and plug the memory stick or flash drive into the adjacent PD machine 20 to install the updated or new treatment prescription. Likewise, treatment data may be downloaded from control unit 30 of PD machine 20 to the memory stick or flash drive 28, which is then plugged into the adjacent docking station 110, where the treatment data is uploaded from the stick to control unit 120 of the docking station, which may then be transported via the remote medical data acquisition and analysis connectivity platform 160 to the appropriate doctor's or clinician's system 170.

Newer PD machines 20 are provided with wireless capability, which allows control units 30 of the newer PD machines to either (i) connect via the Internet 134 directly with the remote medical data acquisition and analysis connectivity platform 160 to share updated or new treatment prescriptions (to PD machine 20) and treatment data (to the doctor or clinician) with the doctor's or clinician's system 170 or (ii) connect wirelessly with docking station 110, which in turn connects via the Internet with the remote medical data acquisition and analysis connectivity platform 160 to share updated or new treatment prescriptions (to PD machine 20) and treatment data (to the doctor or clinician) with the doctor's or clinician's system 170.

It is also contemplated for docking station 110 to be configured to send and/or relay text messages (SMS or MMS messages), e.g., from a doctor's or clinician's cellphone to the patient. In another example, docking station 110 is configured to, under certain treatment or PD machine conditions, relay a wired or wireless alarm sent from PD machine 20 to docking station 110 out to the doctor or clinician cellphone via an SMS or MMS message and/or to a service technician for the PD machine or cycler. Moreover, altering a doctor, clinician and/or technician to a certain treatment or machine, e.g., alarm, condition is expressly not limited to SMS or MMS messaging and may be done via other wired or wireless communication protocols.

Docking station 110 of the present disclosure enables an existing PD machine 20 to be used, with its existing hardware and connectivity, in a system having new advancements in any one or more of (i) medical monitoring, (ii) PD treatment procedures, (iii) data communication, and (iv) data connectivity. Different docking stations 110 may be provided that are customized for the particular age and capability of PD machine 20, different market or regional requirements, and different patient environments. Additionally, new and updated docking stations 110 may be provided as technology advances, while allowing existing and patient familiar cyclers to continue to be used.

PD machine 20 may include and employ batch (tray) heating via a batch heater or inline heating via an inline heater as desired. In various embodiments, the power required for heating PD fluid to treatment, e.g., body temperature of about 37° C., is around 400 Watts for batch (tray) heating and 450 Watts for inline heating. It is contemplated that power supply 118 of docking station 110 is able to meet such power demands plus an added engineering factor above those demands. Such power capability may be provided via a 24 VDC supply at 25 Amps and/or via a 36 VDC supply at 17 Amps (forming a total rating of around 600 Watts. It is also contemplated for docking station 110 and system 10 to use the built-in battery (for backup power) within PD machine 20 to act as a reserve for peaks in power consumption, for example during heating. Docking station 110 may then recharge the built-in battery after the peak power consumption is finished.

Docking station 110 of system 10 in an embodiment provides automated production testing, calibration, and troubleshooting for example. Docking station 110 of system 10 is provided with firewall security to ensure the safe and uncompromised flow of data between the docking station, PD machine 20 and remote medical data acquisition and analysis connectivity platform 160, which may include any one or more of (i) remotely prepared operating software updates delivered to PD machine 20, (ii) remotely prepared patient treatment prescriptions delivered to PD machine 20, (iii) machine and treatment data delivered to remote locations from PD machine 20, (iv) docking station 110 waking PD machine 20 for any needed reason, (v) docking station 110 used to perform remote diagnostics on the patient, and (vi) enabling the patient to remotely start PD machine 20 and begin treatment setup.

If power supply 118 of docking station 110 breaks, PD machine 20 does not have to be sent for repair. A new or backup docking station 110 merely needs to be provided. It is contemplated to provide a smaller and simpler docking station 110 for travel, which may be used as the backup docking station 110 if power supply 118 (or any other component) of the main docking station is broken and while waiting for a new main docking station 110 to be delivered. With a long life-cycle of PD machine 20, the need to adjust to new technologies and new communication protocols may be made by upgrading docking station 110 instead of making changes to or replacing PD machine 20.

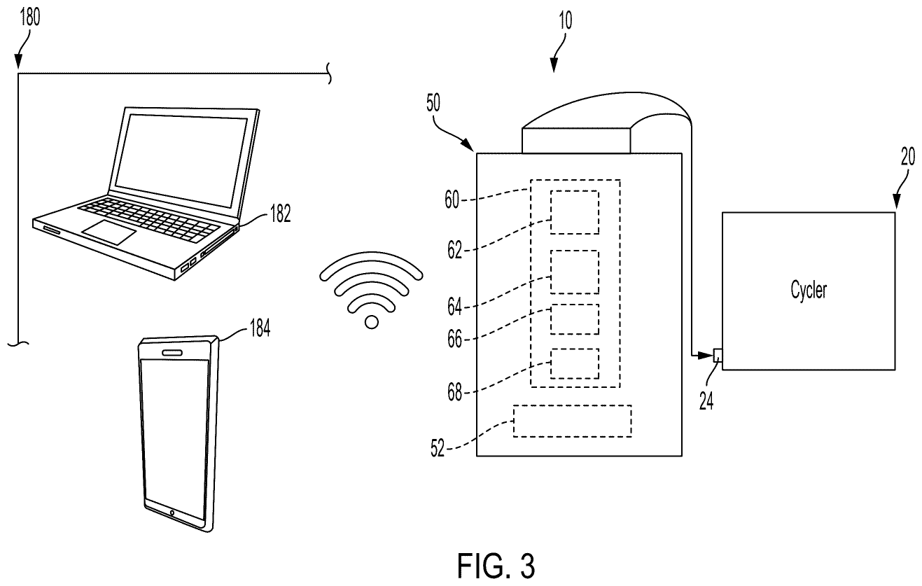

Referring now to FIG. 3, it is further contemplated for system 10 to provide a smart service connector 50, which is plugged into PD machine 20 upon arriving at a service center or depot 180 for repairs, diagnostics or testing. Smart service connector 50 in an embodiment plugs into computer memory or data, e.g., USB, connector/port 26 of PD machine 20 and remains with the PD machine while at service center or depot 180. Smart service connector 50 interfaces with control unit 30 of PD machine 20 so as to provide automatic trouble shooting during storage at service center or depot 180.

In an embodiment, service connector 50 includes a rechargeable battery 52, which is charged by PD machine 20 when the PD machine is plugged into AC power. When PD machine 20 is plugged into AC power, service connector 50 is able to actuate certain components within PD machine 20 to troubleshoot same. For example, service connector 50 is in one embodiment able to cause control unit 30 of PD machine 20 to actuate or toggle the machine's valves, e.g., pneumatic solenoid valves and/or electromechanical fluid pinch valves, to make sure the valves actuate properly. Service connector 50 may also have the ability to cause control unit 30 of PD machine 20 to energize an air pump and to monitor relevant pressure sensors to confirm that target positive and negative pneumatic driving pressures are achievable.

Service connector 50 in the illustrated embodiment includes a control unit 60 including one or more processor 62, one or more memory 64 and a transceiver 66 for wireless communication with service personal computers 182 and/or smart devices 184 located within service center or depot 180. Control unit 60 of service connector 50 may further include a global positioning system (“GPS”) tracker 68 that allows PD machine 20 and the connected service connector 50 to be located at any time within service center or depot 180. Service connector 50 is accordingly able to send interrogation, diagnostic, component testing, PD machine 20 identification and other relevant data to service personal computers 182 and/or smart device 184 for generating work reports and for scheduling further testing and diagnostics for the particular PD machine 20. A work order for the PD machine may be prepared which includes parts needing repair or replacement, further service analysis that needs to be performed, and/or currently working parts that may be refurbished or replaced as a preventative action based on the history of the PD machine. Service connector 50 enables the servicing process to be commenced prior to service personnel actually interrogating the PD machine 20, allowing actions/fixes to be pre-identified and streamlining the servicing process.

It should be understood that various changes and modifications to the presently preferred embodiments described herein will be apparent to those skilled in the art. It is therefore intended that such changes and modifications be covered by the appended claims.

1: A peritoneal dialysis (“PD”) system comprising:
a PD machine configured to provide batch or inline PD fluid heating; and
a docking station connected to the PD machine via a power cord, the power cord bringing power and optionally data to the PD machine, wherein the docking station includes:
a plurality of connectors configured to receive a cable of one or more wired peripheral entity,
a transceiver for wireless communication with one or more wireless peripheral entity, and
a power supply rated to power the PD machine during a PD treatment with at least 600 Watts to support the batch or inline PD fluid heating.

2: The PD system of claim 1, wherein the power cord is the only physical connection between the PD machine and the docking station.

3: The PD system of claim 1, which is configured such that the PD machine is connectable to a plurality of different versions or varieties of the docking station.

4: The PD system of claim 1, which includes the one or more wired and wireless peripheral devices, including at least one medical monitor, the Internet, a network, a smart device, or a cellphone.

5: The PD system of claim 1, wherein the PD machine and the docking station each includes a computer memory connector, a control unit of the PD machine in communication with the PD machine computer memory connector, a control unit of the docking station in communication with the docking station computer memory connector, and wherein a memory stick is used to transport data between the control unit of the PD machine and the control unit of the docking station via the respective computer memory connectors.

6: The PD system of claim 5, wherein the data includes PD treatment data from the PD machine and one or more PD treatment prescription from the docking station.

7: The PD system of claim 6, which includes a clinician and/or hospital system, and wherein the docking station is configured to (i) transmit PD treatment data to the clinician and/or hospital system and (ii) receive the one or more PD treatment prescription from the clinician and/or hospital system.

8: The PD system of claim 7, wherein the docking station is communicatively coupled to the clinician and/or hospital system via at least one of the Internet or a data acquisition and analysis connectivity platform.

9: The PD system of claim 1, wherein the PD machine is configured to communicate wirelessly with the docking station to transport data between the PD machine and the docking station.

10: The PD system of claim 9, wherein the data includes PD treatment data from the PD machine and one or more PD treatment prescription from the docking station.

11: The PD system of claim 10, which includes a clinician and/or hospital system, and wherein the docking station is configured to (i) transmit PD treatment data to the clinician and/or hospital system and (ii) receive the one or more PD treatment prescription from the clinician and/or hospital system.

12: The PD system of claim 1, which includes a clinician and/or hospital system, and wherein the PD machine is configured to communicate wirelessly with the clinician and/or hospital system to transport data between the PD machine and the clinician and/or hospital system.

13: The PD system of claim 12, wherein the data includes PD treatment data from the PD machine and one or more PD treatment prescription from the clinician and/or hospital system.

14: The PD system of claim 12, wherein the PD machine is configured to communicate wirelessly with the clinician and/or hospital system via at least one of the Internet or a data acquisition and analysis connectivity platform.

15: The PD system of claim 1, further including a smart service connector configured to be connected to the PD machine while waiting at a service center for servicing, the smart service connector configured to identify at least one action or fix needed for the PD machine prior to commencing the servicing.

16: The PD system of claim 15, wherein the smart service connector is configured to be plugged into a computer memory connector of the PD machine.

17: The PD system of claim 15, wherein the smart service connector is configured to cause at least one component of the PD machine to be actuated to test the at least one component.

18: The PD system of claim 15, wherein the smart service connector includes a rechargeable battery configured to be recharged by the PD machine.
